(19) United States
(12) Patent Application Publication
(10) Pub. No.: US 2026/0347793 A1
(43) Pub. Date: Nov. 5, 2026

(54) METHOD FOR PRODUCING HIGH-PURITY VASCULAR ENDOTHELIAL CELL POPULATION FROM BIOLOGICAL TISSUE OR ORGAN

(71) Applicants: The University of Osaka, Osaka (JP); Revascular Bio Co., Ltd., Osaka (JP)

(72) Inventors: Nobuyuki Takakura, Osaka (JP); Weizhen Jia, Osaka (JP); Kazuo Omori, Osaka (JP)

(21) Appl. No.: 19/484,780

(22) PCT Filed: May 15, 2024
(86) PCT No.: PCT/JP2024/017958

§ 371 Date: Nov. 14, 2025

(30) Foreign Application Priority Data
May 15, 2023 (JP) 2023-080211

(51) Int. Cl. C12N 5/071; A61K 35/44

(52) U.S. Cl. CPC C12N 5/069; A61K 35/44; C12N 2501/165; C12N 2533/54

(57) ABSTRACT

The present invention provides an invention of a method for producing a vascular endothelial cell population from biological tissue or organ, comprising a step of using a culture vessel configured such that a culture medium in a first culture vessel and a culture medium in a second culture vessel enable to into contact with each other through a porous membrane having pores for cell permeation, wherein a culture surface of the first culture vessel is coated with an extracellular matrix containing a vascular endothelial cell migration factor, and a step of filling the first and second culture vessels with a culture medium, initiating culture of the biological tissue or organ in the second culture vessel, and recovering cells that have permeated through the porous membrane and migrated into the first culture vessel, and an invention of a pharmaceutical composition comprising, as a component, a vascular endothelial cell population produced by the above method.

### TECHNICAL FIELD

The present invention relates to a method for producing a highly purified vascular endothelial cell population from biological tissue or organ, a method for determining an angiogenic potential of a vascular endothelial cell population, a method for purifying vascular endothelial cells, a method for proliferating vascular endothelial cells from biological tissue or organ, and a method for producing vascular endothelial cell sheet.

### BACKGROUND ART

Angiogenesis is induced through two major processes. During fetal development, blood vessels are induced through a process called angiogenesis or vasculogenesis, in which endothelial cells differentiated from mesodermal tissue form tubular structures and construct new vasculature in avascular regions. After birth, a fine vascular network has already been constructed in individual. When new blood vessels are needed due to ischemia or malnutrition, new buds of blood vessels are generated from the existing blood vessels, from which new blood vessels are grown by a process known as angiogenesis. Diseases associated with angiogenesis are collectively known as vascular diseases, and include many conditions, such as malignant tumors, retinopathies, chronic inflammatory diseases, arteriosclerosis, ischemic diseases, diabetes, and autoimmune diseases. Development of new therapies aimed at improving these diseases by controlling angiogenesis are being actively progressed. Therapeutic angiogenesis, which aims to ameliorate disease by regenerating blood vessels, is a therapeutic method for ameliorating the pathology of diseases with reduced blood vessel numbers or impaired vascular function due to various causes by regenerating blood vessels. For example, there are methods for ameliorating ulcers by regenerating blood vessels in ischemic diseases (such as lower limb ischemia and myocardial ischemia) caused by arteriosclerosis and thrombosis, and intractable skin necrosis observed in patients with diabetes or autoimmune diseases.

The effectiveness of therapeutic vascular regeneration for the above-mentioned diseases is being demonstrated through basic medical research using animal models. In actual clinical practice, cell therapy and cytokine (gene) therapy have been applied. For example, the cell therapy includes a method of using endothelial progenitor cells present in bone marrow to regenerate blood vessels. However, because endothelial progenitor cells are not stem cells, the formed blood vessels do not maintain for long periods. Furthermore, the effectiveness of therapeutic method by administrating angiogenesis-inducing cytokines (or their genes) is extremely limited, when used in patients with impaired vascular regeneration capacity.

The present inventors have discovered that vascular endothelial stem cells exist in the existing blood vessels of various organs in the body, which exhibits stem cell properties such as the ability to contribute to the long-term vascularity of transplanted organs and have successfully defined these vascular endothelial stem cells as CD31 (a pan-endothelial cell marker) positive, CD45 (a pan-blood cell marker) negative, CD200 positive, and CD157 positive cells (Non-Patent Document 1). The present inventors also have discovered that these vascular endothelial stem cells can be used to ameliorate ischemia in ischemic diseases for the long term, to reconstruct hepatic sinusoidal vessels, and to completely cure hemophilia by secreting coagulation factor VIII from the reconstructed sinusoidal vessels (Patent Document 1). Furthermore, the present investors discovered that genetic modification of a vascular endothelial cell enables long-term secretion of the transfected molecules in vivo. This finding represents the invention of a new cell therapy approach in which the modified cells function as in vivo biofactories for the continuous production of therapeutic molecules required for the treatment of cancer metabolic diseases and other conditions. (Patent Document 1). The present inventors have also discovered a method for reprogramming human umbilical vein endothelial cells to a vascular endothelial stem cell-like phenotype using an extract of liver tissue from newborn mouse (Patent Document 2).

### Prior Art Documents

### Patent Documents

Patent Document 1: WO2019/098264

Patent Document 2: WO 2021/070874

### Non Patent Documents

Non Patent Document 1: Wakabayashi T. et al., CD157 Marks Tissue-Resident Endothelial Stem Cells with Homeostatic and Regenerative Properties, Cell Stem Cell, 2018; 22(3):384-397.

### SUMMARY OF INVENTION

### Technical Problem

The present invention aims to provide a method for producing a highly purified vascular endothelial cell population from biological tissue or organ, and a pharmaceutical composition comprising the produced vascular endothelial cell population as a component. The present invention also aims to provide a method for determining the angiogenic potential of a vascular endothelial cell population, a method for purifying vascular endothelial cells, a method for proliferating vascular endothelial cells from biological tissue or organ, and a method for producing vascular endothelial cell sheets.

### Solution to Problem

In order to solve the above-mentioned problems, the present invention provides the following aspects.
[1] A method for producing a vascular endothelial cell population from biological tissue or organ, comprising
   a step of using a culture vessel configured such that a culture medium in a first culture vessel and a culture medium in a second culture vessel enable to into contact with each other through a porous membrane having pores for cell permeation,
   wherein a culture surface of the first culture vessel is coated with an extracellular matrix containing a vascular endothelial cell migration factor, and
   a step of filling the first and second culture vessels with a culture medium, initiating culture of the biological tissue or organ in the second culture vessel, and recovering cells that have permeated through the porous membrane and migrated into the first culture vessel.
[2] The method according to [1], wherein the purity of vascular endothelial cells in the vascular endothelial cell population is 50% or higher.
[3] The method according to [1] or [2], wherein the proportion of CD157-positive cells among the vascular endothelial cells in the vascular endothelial cell population is 5% or higher.
[4] The method according to any one of [1] to [3], wherein the porous membrane is formed from a biocompatible material.
[5] The method according to [4], wherein the biocompatible material is selected from the group consisting of collagen, fibrin, hyaluronic acid, polylactic acid, polyglycolic acid, polycaprolactone, polydioxanone, trimethylene carbonate, polyethylene glycol, alginate, chitosan, and mixtures thereof.
[6] The method according to any one of [1] to [5], wherein the vascular endothelial cell migration factor is one or more selected from the group consisting of vascular endothelial cell growth factor, basic fibroblast growth factor, hepatocyte growth factor, epidermal growth factor, SDF-1, granulocyte colony-stimulating factor, leukemia inhibitory factor, nerve growth factor, platelet-derived growth factor, stem cell growth factor, axon repulsive factor, granulocyte-macrophage colony-stimulating factor, sphingosine-1-phosphate, angiopoietin, interleukins, CCL5, CCL1, placenta growth factor, apelin, elabella, netrin, semaphorin, gremlin, BafA, and serum.
[7] The method according to any one of [1] to [6], wherein the extracellular matrix is one or more selected from the group consisting of basement membrane matrix, laminin, type I collagen, type IV collagen, fibronectin, vitronectin, fibrin, tenascin, hyaluronic acid, and proteoglycan.
[8] A pharmaceutical composition comprising, as a component, a vascular endothelial cell population produced by the method according to any one of [1] to [7].
[9] The pharmaceutical according to [8], which is for use in regenerating blood vessels, ameliorating ischemia, ameliorating malnutrition, treating vascular malformations, ameliorating blood flow insufficiency caused by vascular malformations, promoting organ regeneration, or treating diseases caused by abnormalities in molecules secreted from vascular endothelial cells.
[10] A method for determining an angiogenic potential of a vascular endothelial cell population produced by the method according to any one of [1] to [7], comprising
a step of placing a second culture vessel having the porous membrane on its bottom surface on top of the first culture vessel and initiating culture; and
a step of determining that the resulting vascular endothelial cell population has angiogenic potential in the case where the cells on the bottom surface of the lower culture vessel of the first culture vessel are observed after the start of culture, and a vascular network is formed.
[11] A method for purifying vascular endothelial cells from biological tissue or organ, comprising
   a step of using a culture vessel configured such that a culture medium in a first culture vessel and a culture medium in a second culture vessel enable to into contact with each other through a porous membrane having pores for cell permeation,
   wherein a culture surface of the first culture vessel is coated with an extracellular matrix containing a vascular endothelial cell migration factor, and
   a step of filling the first and second culture vessels with a culture medium, initiating culture of the biological tissue or organ in the second culture vessel, and recovering cells that have permeated through the porous membrane and migrated into the first culture vessel.
[12] The method according to [11], wherein the purity of vascular endothelial cells in the recovered cells is 50% or higher.
[13] A method for proliferating vascular endothelial cells from biological tissue or organ, comprising:
   a step of using a culture vessel configured such that a culture medium in a first culture vessel and a culture medium in a second culture vessel enable to into contact with each other through a porous membrane having pores for cell permeation,
   wherein a culture surface of the first culture vessel is coated with an extracellular matrix containing a vascular endothelial cell migration factor; and
   a step of filling the first and second culture vessels with a culture medium and culturing the biological tissue or organ in the second culture vessel.
[14] A method for producing a vascular endothelial cell sheet, comprising:
   a step of using a culture vessel configured such that a culture medium in a first culture vessel and a culture medium in a second culture vessel enable to into contact with each other through a porous membrane having pores for cell permeation;
   wherein a culture surface of the first culture vessel is coated with an extracellular matrix containing a vascular endothelial cell migration factor;
   a step of filling the first and second culture vessels with a culture medium, initiating culture of the biological tissue or organ in the second culture vessel; and
   a step of recovering the porous membrane and removing the cells and tissue or organ present on the porous membrane on the second culture vessel side.
[15] The method according to [14], wherein the porous membrane is formed from a biocompatible material.

### Effects of Invention

The present invention can provide a method for producing a highly purified vascular endothelial cell population from biological tissue or organ, and a pharmaceutical composition comprising the produced vascular endothelial cell population as a component. The present invention can also provide a method for determining the angiogenic potential of a vascular endothelial cell population, a method for purifying vascular endothelial cells, a method for proliferating vascular endothelial cells from biological tissue or organ, and a method for producing vascular endothelial cell sheets.

### BRIEF DESCRIPTION OF DRAWINGS

FIG. 1 shows the experimental procedure of Example 1.

FIG. 2 (A) shows a representative image of cells adhered to the underside of the porous membrane (polycarbonate membrane) of the upper culture vessel (Transwell insert) immunostained with anti-CD31 antibody, and FIG. 2 (B) shows a representative image of cells on a basement membrane matrix coated on the bottom of the well.

FIG. 3 shows the results of flow cytometry analysis of cells before and after culture, by staining surface antigens with anti-CD31, anti-CD45, and anti-CD157 antibodies. FIG. 3 (A) shows the results of cells obtained by dispersing adipose tissue before the start of culture, FIG. 3 (B) shows the results of cells recovered from the underside of the porous membrane on day 14 of culture, and FIG. 3 (C) shows the results of cells recovered from the basement membrane matrix on day 14 of culture.

FIG. 4 shows the experimental procedure of Comparative Example 1.

FIG. 5 shows a representative image of vascular endothelial cells isolated from human adipose tissue and human mesenchymal stem cells, which are co-cultured for 10 days and immunostained with anti-CD31 antibody.

FIG. 6 shows the experimental procedure for Comparative Example 2.

FIG. 7 shows a representative image and an enlarged image of cells that have expanded and proliferated from minced human adipose tissue after 21 days of culture, which are immunostained with anti-CD31 antibody.

FIG. 8 shows the results of flow cytometry analysis of cells that have expanded and proliferated from minced human adipose tissue after 21 days of culture, which were recovered by mechanical and enzymatic dispersion, by staining using anti-CD44 antibody, or anti-CD31 antibody and anti-CD45 antibody for surface antigen. FIG. 8 (A) shows the results of analyzing the ratio of CD44-positive cells (mesenchymal cells), and FIG. 8 (B) shows the results of analyzing the ratio of CD31-positive, CD45-negative cells (vascular endothelial cells).

FIG. 9 shows the results of confirming the wound healing effect of a vascular endothelial cell sheet. FIG. 9 (A) is images of the back of nude mice immediately after skin removal and 7 days after application of a vascular endothelial cell sheet, and (B) is a graph showing the recovery rate of the skin injury site.

FIG. 10 shows the results of histological observation of the vascular endothelial cell sheet-application site. FIG. 10 (A) is the result of a control mouse, and FIG. 10 (B) is the result of a mouse with a vascular endothelial cell sheet applied.

FIG. 11 shows a representative image of cells adhered to the underside of the collagen membrane of the upper culture vessel (cell strainer) immunostained with an anti-CD31 antibody.

### DESCRIPTION OF EMBODIMENTS

### Method for Producing Vascular Endothelial Cell Population

The present invention provides a method for producing a vascular endothelial cell population from biological tissue or organ (hereinafter referred to as the “production method of the present invention”). Using the production method of the present invention, a highly purified vascular endothelial cell population can be produced from biological tissue or organ.

The biological tissue or organ used in the production method of the present invention is not particularly limited as long as it contains vascular endothelial cells. Examples include placenta, umbilical cord, skin, liver, lung, brain, kidney, omentum, muscle, intestinal mucosa, gastric mucosa, and adipose tissue.

The production method of the present invention can be carried out using a culture vessel configured to allow contact between the medium in the first culture vessel and the medium in the second culture vessel via a porous membrane having pores for cell permeation. Examples of culture vessels with such a structure include, but are not limited to, a culture vessel in which a second culture vessel with a porous membrane on its bottom surface is placed within the first culture vessel (see, for example, FIG. 1).

When using a culture vessel in which a second culture vessel with a porous membrane on its bottom surface is placed within the first culture vessel, a cell culture dish, a cell culture multi-well plate, or the like can be used as the first culture vessel. The size of the culture vessel is not particularly limited and can be selected appropriately depending on the number of cells of the vascular endothelial cell population to be produced. The second culture vessel is not particularly limited as long as it can be placed within the first culture vessel and has a porous membrane on its bottom surface. It can be a commercially available product or a homemade product. Examples of commercially available products include Transwell (registered trademark) Permeable Support (Corning Incorporated), TC Insert (SARSTEDT), Millicell (registered trademark) Cell Culture Insert (MILLIPORE), etc. Examples of homemade products include those in which the mesh of a commercially available cell strainer is removed and any porous membrane is attached to the bottom.

The material of the porous membrane is not particularly limited as long as it is compatible with cell culture. Examples include polycarbonate, polyester (PET), polytetrafluoroethylene, polysulfone, polypropylene, polyvinylidene fluoride, and polyamide. The porous membrane may be formed from a biocompatible material. Biocompatible materials are not particularly limited, but, for example, include collagen, fibrin, hyaluronic acid, polylactic acid, polyglycolic acid, polycaprolactone, polydioxanone, trimethylene carbonate, polyethylene glycol, alginate, chitosan, and mixtures thereof. The pore size of the porous membrane may be large enough to allow vascular endothelial cells to pass through. For example, the pore size may be 1 μ(micro)m or more, 3 μ(micro)m or more, 5 μ(micro)m or more, 8 μ(micro)m or more, or 10 μ(micro)m, or 20 μ(micro)m or less, 15 μ(micro)m or less, or 12 μ(micro)m or less.

The first culture vessel used in the production method of the present invention has a culture surface coated with an extracellular matrix containing a vascular endothelial cell migration factor. The extracellular matrix is not particularly limited, but, for example, includes basement membrane matrix, laminin, type I collagen, type IV collagen, fibronectin, vitronectin, fibrin, tenascin, hyaluronic acid, and proteoglycan. Commercially available basement membrane matrices, for example, include “Cultrex Basement Membrane Extract (BME)” manufactured by R&D Systems and “Matrigel Basement Membrane Matrix” manufactured by Corning Incorporated.

Examples of vascular endothelial cell migration factors include, for example, vascular endothelial growth factor (VEGF), basic fibroblast growth factor (bFGF), hepatocyte growth factor (HGF), epidermal growth factor (EGF), SDF-1 (stromal cell-derived factor-1), granulocyte colony stimulating factor (G-CSF), leukemia inhibitory factor (LIF), nerve growth factor (NGF), platelet-derived growth factor (PDGF), stem cell growth factor (SCF), axonal repulsion factor (Slit), granulocyte-macrophage colony stimulating factor (GM-CSF), sphingosine-1-phosphate (S1P), angiopoietin (ANGPT), interleukin (IL), CCL5 (C-C motif chemokine ligand 5, also known as RANTES), CCL1 (C-C motif chemokine ligand 1, also known as 1-309), placental growth factor (PLGF), apelin, Elabella, netrin, semaphorin, and serum. Agonists of the receptors for the vascular endothelial cell migration factors listed above can also be suitably used as vascular endothelial cell migration factors. Examples of agonists for the VEGF receptor include Gremlin and BafA.

The amount of vascular endothelial cell migration factor added to the extracellular matrix can be appropriately selected depending on the combination of extracellular matrix and vascular endothelial cell migration factor used. For example, when VEGF is used as the vascular endothelial cell migration factor and basement membrane matrix is used as the extracellular matrix, the VEGF concentration in the extracellular matrix is preferably 1 ng/ml to 1000 ng/mL.

The production method of the present invention uses such a culture vessel, fills the first and second culture vessels with culture medium, initiates the culture of a biological tissue or organ in the second culture vessel, and recovers cells that have migrated through the porous membrane into the first culture vessel. Any culture medium capable of culturing vascular endothelial cells may be used, including known culture medium that has been used to culture vascular endothelial cells. For example, commercially available culture medium such as HuMedia-EB2 manufactured by Kurabo Industries Ltd., EGM-2MV manufactured by LONZA, and MiraCell EC Culture Medium manufactured by Takara Bio Inc. can be used. When the obtained vascular endothelial cell population is to be used for human treatment, it is preferable to use a xeno-free culture medium.

The biological tissue or organ to be cultured in the second culture vessel is preferably placed in contact with the porous membrane. Furthermore, it is preferable to mince the tissue or organ to promote cell expansion and proliferation. The size of the minced tissue block is not particularly limited, but may be, for example, a 1 mm-10 mm cube. The amount of tissue to be cultured can be appropriately determined taking into account of the size of the second culture vessel and the area of the porous membrane.

Culture can be carried out under conditions typically used for cell culture (e.g., 37 °C, 5% CO₂). The culture period is not particularly limited. The end of culture is preferably determined by observing the state of cells that have permeated the porous membrane and adhered to the porous membrane surface on the first culture vessel side, and/or the state of cells on the culture surface of the first culture vessel. The state of cells is preferably determined by observing colony formation and angiogenesis.

The method for recovering cells migrated into the first culture vessel is not particularly limited. For example, the cells migrated into the first culture vessel can be recovered by using a known cell release agent to detach the cells adhered to the porous membrane surface of the first culture vessel and the cells adhered to the culture surface of the first culture vessel. If the recovered cells are CD31-positive or CD31-positive CD45-negative, they can be determined to be vascular endothelial cells. Furthermore, vascular endothelial stem cells among the recovered cells can be confirmed by the presence of CD31-positive CD157-positive cells or CD31-positive CD45-negative CD157-positive cells.

The production method of the present invention can obtain a vascular endothelial cell population with purity of 50% or higher of vascular endothelial cells. The purity of the vascular endothelial cells may be 60% or higher, 70% or higher, 80% or higher, 90% or higher, 95% or higher, 96% or higher, 97% or higher, 98% or higher, or 99% or higher. The production method of the present invention makes it possible to obtain a vascular endothelial cell population in which the proportion of vascular endothelial stem cells (CD157-positive cells) among the obtained vascular endothelial cells is 5% or higher. The ratio of vascular endothelial stem cells among the vascular endothelial cells may be 6% or higher, 7% or higher, 8% or higher, 9% or higher, or 10% or higher.

The vascular endothelial cell population obtained by the production method of the present invention significantly increases the ratio of the vascular endothelial cell content in the biological tissue or organ subjected to the production method of the present invention, thereby increasing the number of vascular endothelial cells. Therefore, the present invention provides a method for purifying vascular endothelial cells from biological tissue or organ. The present invention also provides a method for proliferating vascular endothelial cells of biological tissue or organ.

### Pharmaceutical Composition

The present invention provides a pharmaceutical composition comprising, as a component, a vascular endothelial cell population produced by the above-described production method of the present invention. The pharmaceutical composition of the present invention can be used for various medical purposes, including vascular regeneration, amelioration of ischemia, amelioration of malnutrition, treatment of vascular malformations, amelioration of blood flow insufficiency caused by vascular malformations, promotion of organ regeneration, and treatment of diseases caused by abnormalities in molecules secreted by vascular endothelial cells.

The vascular endothelial cell population of the present invention comprises vascular endothelial stem cells. The inventors have confirmed that vascular endothelial stem cells transplanted into the liver of a mouse model of hepatic vascular disorder results in vascular regeneration (see, for example, Patent Document 1). Therefore, the pharmaceutical composition of the present invention can be suitably used as a pharmaceutical composition for vascular regeneration. The pharmaceutical composition of the present invention can ameliorate ischemia and malnutrition caused by impaired vascular function by regenerating blood vessels. Therefore, the pharmaceutical composition of the present invention is effective in treating ischemic diseases such as cerebral infarction, myocardial infarction, and Buerger's disease. These ischemic diseases may be caused by arterial diseases such as arteriosclerosis, thrombosis, and arteritis, or by lifestyle-related diseases such as hyperlipidemia, diabetes, hypertension, gout, and aging. Furthermore, the pharmaceutical composition of the present invention can be appropriately used for the treatment of vascular malformations or blood flow insufficiency caused by vascular malformations. Vascular malformations include arteriovenous fistulas and moyamoya disease. Furthermore, the pharmaceutical composition of the present invention can also be used for wound healing.

The vascular endothelial cell population of the present invention comprises vascular endothelial stem cells. The inventors have confirmed that vascular endothelial stem cells transplanted into the liver of a mouse undergoing 70% partial hepatectomy promotes liver regeneration (see, for example, Patent Document 1). Therefore, the pharmaceutical composition of the present invention can be used to promote organ regeneration. The target organ is not particularly limited; the pharmaceutical composition of the present invention can promote regeneration of any organ as long as vascular regeneration is possible. It has been demonstrated that organ-specific cells (including stem cells) in any organ are induced to prolong cell survival and proliferation by humoral factors and adhesion factors secreted by vascular endothelial cells. Therefore, organ regeneration is promoted in association with the regeneration of the organ's blood vessels. Therefore, the pharmaceutical composition of the present invention can treat diseases that are improved by organ regeneration. For example, promoting liver regeneration can enable the prevention or treatment of liver cirrhosis, hepatic fibrosis, hepatitis, fatty liver, and liver failure. The same applies to other organs.

The vascular endothelial cell population of the present invention includes vascular endothelial stem cells. The inventors have confirmed that administering the cell population of the present invention, prepared from the liver of a mouse with a normal coagulation factor VIII gene, to the liver of a hemophilia A model mouse significantly shortened the time from bleeding to hemostasis (see, for example, Patent Document 1). Therefore, the pharmaceutical composition of the present invention can be used to treat diseases caused by abnormalities in molecules secreted from vascular endothelial cells. Diseases caused by abnormalities in molecules secreted from vascular endothelial cells include, for example, hemophilia A (coagulation factor VIII), hemophilia B (coagulation factor IX), lysosomal storage diseases (alpha-galactosidase), von Willebrand disease (von Willebrand factor), hypertension (nitric oxide, etc.), impaired glucose tolerance (nitric oxide, etc.), dyslipidemia (nitric oxide, etc.), metabolic syndrome (apelin, etc.), and osteoporosis (Notch ligand, netrin, endothelin-1, etc.) (the molecules secreted from vascular endothelial cells are indicated in parentheses).

The pharmaceutical composition of the present invention can be administered to a living body in the form of a cell suspension in which the vascular endothelial cell population of the present invention is suspended in an appropriate solution suitable for administration to the living body. Examples of suitable solutions include physiological saline, phosphate-buffered saline (PBS), and other physiological salt solutions. The vascular endothelial cell population of the present invention is typically prepared immediately prior to administration, but may also be prepared immediately before use using a cryopreserved vascular endothelial cell population. The pharmaceutical composition of the present invention can be administered by administering the vascular endothelial cell population of the present invention directly to the target organ for revascularization or intravenously upstream of the target organ for revascularization. The dosage varies depending on the target organ for revascularization, the patient's age and body weight, and therefore cannot be determined unambiguously. However, a physician can determine an appropriate dosage based on the above-mentioned conditions. For example, 1 to 1×10⁹ cells may be administered per administration. The frequency of administration can be selected as appropriate from once per day to once per week. The dosage and frequency of administration can be increased or decreased as appropriate for the patient.

Method for Determining the Angiogenic Potential of a Vascular Endothelial Cell Population The present invention provides a method for determining the angiogenic potential of a vascular endothelial cell population produced by the above-described production method of the present invention. The inventors have performed the production method of the present invention by placing a second culture vessel having the porous membrane on its bottom surface on top of a first culture vessel. The inventors have confirmed that when vascular endothelial cells form colonies on the porous membrane surface on the side of the first culture vessel (i.e., underside of the porous membrane), a vascular network structure is observed on the bottom surface of the first culture vessel (on the coated basement membrane matrix) (see Example 1 of the present application). Therefore, when the production method of the present invention is performed by placing a second culture vessel having the porous membrane on its bottom surface on top of a first culture vessel, if a vascular network is formed by observing the cells on the bottom surface of the first culture vessel after the start of culture, the resulting vascular endothelial cell population can be determined to have angiogenic potential.

### Method for Producing a Vascular Endothelial Cell Sheet

The present invention provides a method for producing a vascular endothelial cell sheet. The method for producing a vascular endothelial cell sheet of the present invention can be carried out by recovering the porous membrane and removing the cells and tissues or organs present on the porous membrane on the second culture vessel side, in place of recovering the cells in the first culture vessel after the start of culture in the above-described production method of the present invention. Since vascular endothelial cells that have permeated the porous membrane adhere to the surface of the porous membrane on the side of the first culture vessel, the porous membrane can be used as a vascular endothelial cell sheet as is.

The porous membrane is preferably made from a biocompatible material. Biocompatible materials are not particularly limited, but examples include collagen, fibrin, hyaluronic acid, polylactic acid, polyglycolic acid, polycaprolactone, polydioxanone, trimethylene carbonate, polyethylene glycol, alginate, chitosan, and mixtures thereof.

A vascular endothelial cell sheet produced by the method for producing a vascular endothelial cell sheet of the present invention can be used to repair the damage by applying to damaged areas of the skin. The vascular endothelial cell sheet can also be used to improve diseases that develop or worsen due to ischemia by applying to areas experiencing ischemia. The site of application is not limited as long as it is an area where ischemia occurs. For example, the sheet can be applied to the brain for cerebral ischemia, to the heart for myocardial ischemia, or to around blood vessels in the lower limbs for lower limb ischemia. The vascular endothelial cell sheet can also be used to improve diseases that develop or worsen due to vascular damage or abnormalities by applying to areas experiencing vascular damage or abnormalities.

Diseases that can be improved with a vascular endothelial cell sheet include, but are not limited to, dementia/Alzheimer's disease, moyamoya disease, heart failure, liver fibrosis, liver cirrhosis, scleroderma, diabetic gangrene, autoimmune diseases, interstitial pneumonia, chronic obstructive pulmonary disease, pulmonary hypertension, glomerulonephritis, kidney disease, radiation sequelae, and malignant tumors.

### EXAMPLES

The present invention will be described in more detail below with reference to Examples, but the present invention is not limited thereto.

### Example 1: Organ Culture of Minced Adipose Tissue Using a Porous Membrane Materials and Methods

### Culture of Human Adipose Tissue

The procedure of Example 1 is shown in FIG. 1. Human adipose tissue obtained by surgery was minced with scissors to prepare cubic minced tissue blocks with sides of approximately 2 mm. A 24-well Costar cell culture multiwell plate (Corning Incorporated) was used as the culture vessel, with a Transwell insert (Corning Incorporated) equipped with an 8.0 μ(micro)m pore-size polycarbonate membrane inserted. Thawed basement membrane matrix (Cultrex Basement Membrane Extract (BME) from R&D Systems) was supplemented with VEGF (PEPRO TECH) at a final concentration of 400 ng/ml. 250 μ(micro)L of this solution was dispensed into each well of a 24-well plate and allowed to solidify at 37 °C. 250 μ(micro)L of Hu-Media EG2 (serum-free liquid culture medium for normal human vascular endothelial cell growth, Kurabo) was then added. Minced adipose tissue was placed on the polycarbonate membrane of a Transwell insert, which was inserted into each well of the 24-well plate. 80 μ(micro)L of Hu-Media EG2 was added to the insert, and the cells were cultured at 37 °C in 5% CO₂. The culture medium was replaced on days 6 and 12. On day 14 of culture, the inserts were recovered for immunostaining and flow cytometry analysis.

### (2) Immunostaining of Cells on the Underside of the Membrane and Observation of Cells on the Matrix

To detect vascular endothelial cells on the underside of the polycarbonate membrane, the polycarbonate membrane was excised from the insert and fixed with 4% paraformaldehyde. An anti-CD31 antibody (clone WM59, BioLegend) was used to perform immunostaining and diaminobenzidine (DAB, DOJINDO) was used to perform color development. Cells on the matrix coating on the bottom of the well were observed using a phase-contrast microscope.

### (3) Flow Cytometry Analysis

For flow cytometry analysis, the polycarbonate membrane was excised from the insert. The adipose tissue and cells on the upper surface of the membrane were removed with a cotton swab. The cells on the lower surface were washed with PBS and then detached and collected using Accutase (Innovative Cell Technologies, Inc.). An anti-CD31 antibody (clone WM59, BioLegend), an anti-CD45 antibody (clone 2D1, BioLegend), and an anti-CD157 antibody (clone RF3, MBL Life Sciences) were used to perfom cell surface antigen staining and a FACSAria II was used to perform analysis.

### 2. Results

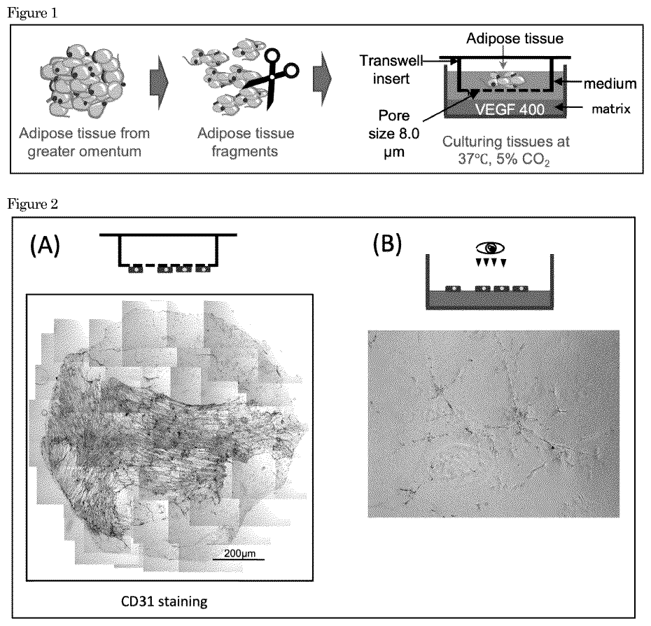

A representative image of stained vascular endothelial cells (CD31-positive cells) on the underside of the polycarbonate membrane is shown in FIG. 2(A). On the upper surface of the polycarbonate membrane, mesenchymal cells, expanded from adipose tissue, proliferated, as in Comparative Example 2 described below. However, vascular endothelial cells were observed to pass through the pores of the porous membrane according to the concentration gradient of VEGF contained in the basement membrane matrix at the bottom of the well, forming very large colonies up to approximately 1 cm in diameter on the underside of the porous membrane. These results suggest that vascular endothelial cells are proliferating and are is produced from vascular endothelial stem cells.

A representative image of cells on the matrix coating on the bottom of the well is shown in FIG. 2(B). A vascular network structure was observed on the coated basement membrane matrix, and it was confirmed that vascular endothelial cells that passed through the pores of the porous membrane further migrated into the basement membrane matrix and formed a vascular network on the matrix. Whenever vascular endothelial cells formed colonies on the underside of the porous membrane, a vascular network structure was observed on the matrix below. Therefore, if a vascular network was confirmed to have formed on the matrix, it was believed that the cells adhered to the underside of the porous membrane and/or the vascular endothelial cells present in the lower culture vessel could be guaranteed to have angiogenic potential.

The results of flow cytometry analysis are shown in FIG. 3. FIG. 3 (A) shows the results of cells obtained by dissociating adipose tissue before the start of culture, FIG. 3 (B) shows the results of cells recovered from the underside of a porous membrane, and FIG. 3 (C) shows the results of cells recovered from a basement membrane matrix. Before the start of culture of FIG. 3 (A), adipose tissue contained 2.8% CD31-positive vascular endothelial cells, in which CD157-positive vascular endothelial stem cells were only 2.8% among them. All cells recovered from the underside of a porous membrane of FIG. 3 (B) were CD31-positive vascular endothelial cells, in which CD157-positive vascular endothelial stem cell fractions were 14.8% among them. All cells recovered from a basement membrane matrix of FIG. 3 (C) were also CD31-positive vascular endothelial cells, in which CD157-positive vascular endothelial stem cell fractions were 6.5% among them. These results demonstrate that the culture method of this example allows for the purification of vascular endothelial cells without the need of specific cell fractionation procedures such as fluorescence spectroscopy or magnetic cell sorting. Furthermore, the culture method of this example increases the ratio of vascular endothelial stem cells (CD157 positive) among the purified vascular endothelial cells.

### Comparative Example 1: Co-Culture of Adipose Tissue-Derived Vascular Endothelial Cells and Mesenchymal Stem Cells

### Materials and Methods

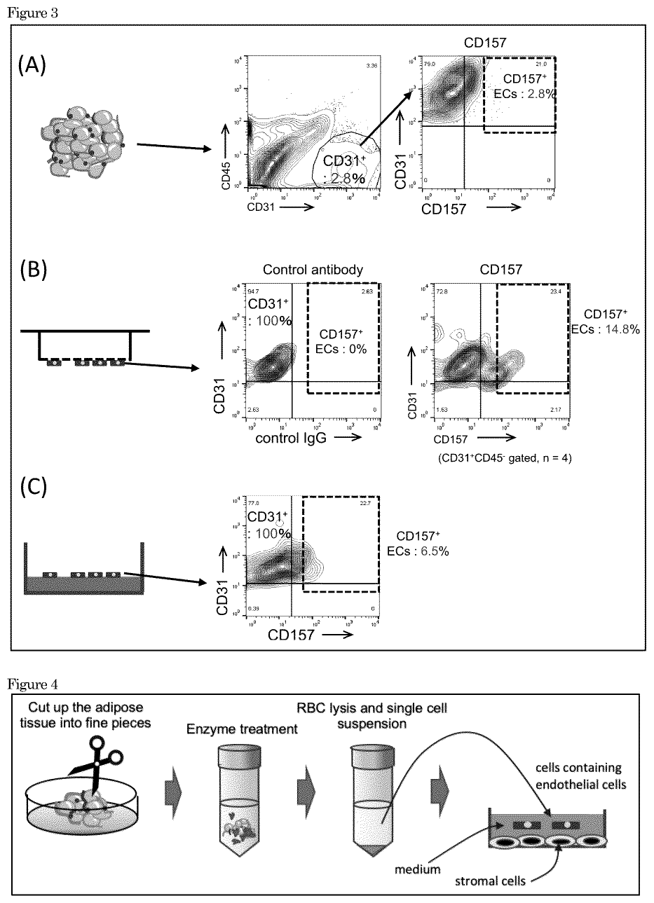

The procedure of Comparative Example 1 is shown in FIG. 4. Human adipose tissue obtained by surgery was minced with scissors. A collagenase-based enzyme solution (a solution of 4% fetal bovine serum (FBS) in PBS (4% FBS/PBS) supplemented with 0.2% type I collagenase (FUJIFILM Wako Pure Chemical Corporation), 3 mM CaCl₂ (FUJIFILM Wako Pure Chemical Corporation), and 20 μ(micro)g/mL DNase I (F. Hoffmann-La Roche Ltd)) was added to the resulting sludge. The mixture was then shaken on a rotator at 37 °C for 30 minutes to digest the extracellular matrix. Cells were collected by centrifugation, and any remaining red blood cells were lysed using red blood cell lysing buffer (Sigma-Aldrich). After hemolysis, the cells were washed with PBS containing 4% FBS and collected by centrifugation. The obtained adipose tissue-derived cells were subjected to surface antigen staining using an anti-CD31 antibody (clone WM59, BioLegend) and an anti-CD45 antibody (clone 2D1, BioLegend), and vascular endothelial cells (CD31 positive, CD45 negative) were isolated by flow cytometry using a FACSAria II (Becton, Dickinson and Company).

Human mesenchymal stem cells (hMSCs, Lonza Japan Ltd.) were used as stromal cells. Mesenchymal stem cells were cultured in a 6-well culture plate (Thermo Scientific) until confluent. Isolated adipose tissue-derived vascular endothelial cells were seeded on top of the mesenchymal stem cells at 2×10³ cells/well and co-cultured in Hu-Media EG2 containing 20 ng/mL VEGF at 37 °C and 5% CO₂. 60 ng of VEGF was added every 3 days during the culture period. On day 10 of culture, the culture medium was removed and the cells were fixed with 4% paraformaldehyde. To detect vascular endothelial cells, an anti-CD31 antibody (clone WM59, BioLegend) was used to perform immunostaining and diaminobenzidine (DAB, DOJINDO) was used to perform color development.

### 2. Results

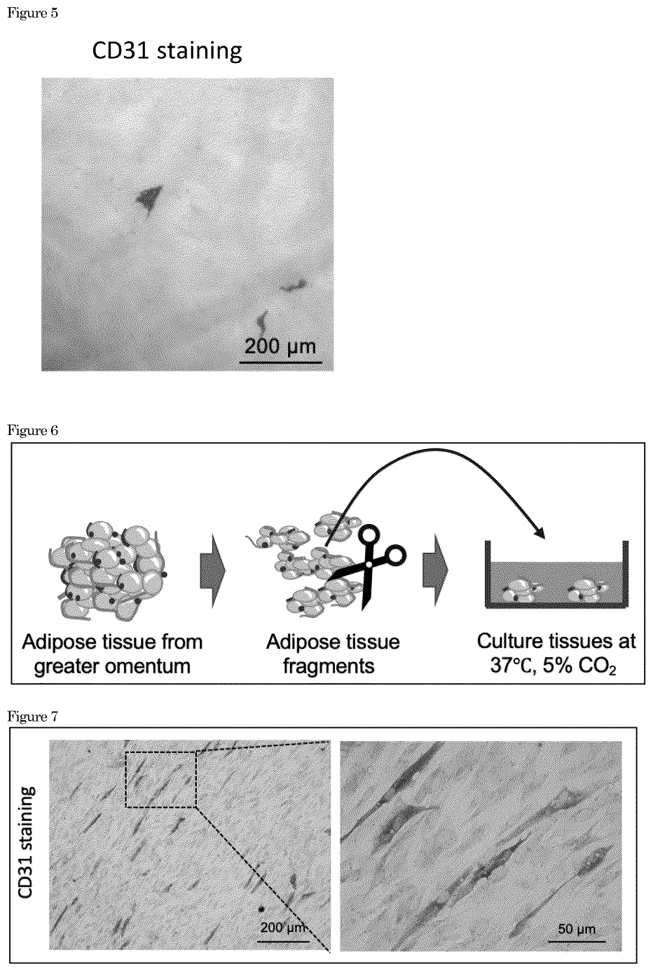

A representative image of stained vascular endothelial cells (CD31-positive cells) on day 10 of coculture is shown in FIG. 5. When mesenchymal stem cells were co-cultured as stromal cells, the size of the observed vascular endothelial cell colonies were less than 50 μ(micro)m in diameter, and no large vascular endothelial cell colonies according to vascular endothelial cell proliferation were observed.

### Comparative Example 2: Detection of Vascular Endothelial Cells by Organ Culture of Minced Adipose Tissue

### Materials and Methods

The procedure of Comparative Example 2 is shown in FIG. 6. Human adipose tissue obtained by surgery was minced with scissors into cubes with sides of approximately 2-6 mm. The cubes were placed on a 3 cm diameter culture dish, immersed in Hu-Media EG2, and cultured at 37 °C with 5% CO₂. Microscopic observations were performed over time. The culture medium was changed every 7 days. On day 21 of culture, the culture medium was removed, and the tissue and cells on the culture dish were fixed with 4% paraformaldehyde. To detect vascular endothelial cells, an anti-CD31 antibody (clone WM59, BioLegend) was used to perform immunostaining and diaminobenzidine (DAB, DOJINDO) was used to perform color development. Separately, live cells were collected from other culture dishes by mechanical and enzymatic dispersion and an anti-CD31 antibody (clone WM59, BioLegend), an anti-CD45 antibody (clone 2D1, BioLegend), and an anti-CD44 antibody (clone BJ18, BioLegend) were used to perform surface antigen staining, followed by flow cytometry analysis using a FACSAria II.

### 2. Results

The results of immunostaining CD31-positive cells on day 21 of culture are shown in FIG. 7. The image on the right is an enlarged image of the area in the left frame. Spindle-shaped cells expanded and proliferated from the adipose tissue, gradually covering the entire periphery of the adipose tissue. Some of the cells were CD31-positive vascular endothelial cells. However, these vascular endothelial cells gradually decreased over the course of culture. No vascular endothelial cell colonies were observed.

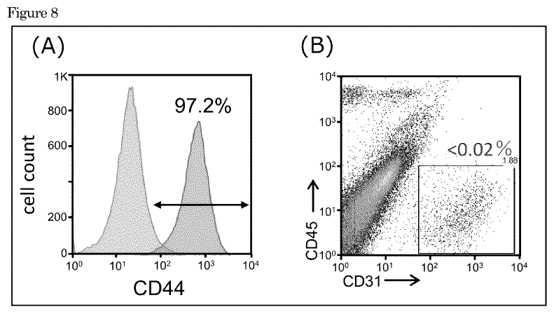

Tissues and cells on the surface of the culture dish were dispersed into single cells on day 21 of culture, and the results of flow cytometry analysis are shown in FIG. 8. Most of the cells were CD44-positive mesenchymal cells, and the presence of vascular endothelial cells (CD31-positive, CD45-negative) was less than 0.02%.

### Example 2: Study of Wound Healing Using Vascular Endothelial Cells Obtained by the Method of Example 1

In organ regeneration, tissue regeneration is not possible unless blood vessels that transport oxygen and nutrients are formed. In other words, enhancing angiogenesis induces improvement of blood flow, accelerates the supply of oxygen and nutrients, and promotes rapid tissue recovery. Conventional treatments for vascular regeneration have used bone marrow cells and mesenchymal stem cells. However, since these cells themselves lack the ability to contribute to vascularization, or are extremely rare, treatment is limited to relying on the paracrine effect of angiogenesis-inducing substances secreted by the cells. In such treatments, neovascularization cannot be expected in patients with low reserve capacity for angiogenesis. On the other hand, transplanting and establishing a cell fraction of vascular endothelial stem cells capable of forming blood vessels into organs requiring revascularization is expected to lead to more rapid organ regeneration. Therefore, we investigated whether the presence of vascular endothelial stem cells induces angiogenesis and promotes wound healing during the course of wound healing in mice.

### Materials and Methods

KSN nude mice (Japan SLC Co., Ltd.) were used to transplant human vascular endothelial cells. A wound model was created by removing a 3 mm diameter piece of skin, including the dermis, from the back of each mouse using a Derma Punch 3 mm (Maruho Co., Ltd.). Adipose tissue was cultured by the same method as in Example 1, and the polycarbonate membrane was excised from the Transwell insert. The adipose tissue and cells on the upper surface of the membrane were removed with a cotton swab, creating a sheet containing only endothelial cells, including vascular endothelial stem cells, remaining on the underside surface of the membrane. This sheet was applied to the damaged skin site, and a Tegaderm (3M Co., 1622W) sheet was then applied over it for fixation and observation. The sheet was removed on day 5, and subsequent skin recovery was observed. Mice with the polycarbonate membrane of a Transwell insert without adipose tissue culture applied to the damaged skin site served as controls.

For histological analysis, skin samples were collected from the injury site, fixed in 4% paraformaldehyde, and embedded in Tissue-Tec O.C.T. Compound (Sakura Finetech Japan) to prepare frozen blocks. The entire skin area was then sliced at 10 μ(micro)m thickness. The sections were stained with hematoxylin and eosin using standard methods and subjected to histological analysis. Immunostaining was performed to analyze the extent to which human adipose tissue-derived vascular endothelial cells contribute to angiogenesis in mouse skin tissue. For immunostaining, a rat anti-mouse CD31 antibody (clone MEC13.3, BD Biosciences) and a rabbit anti-human CD31 antibody (clone EPR3094, Abcam) were used as primary antibodies, and Alexa Fluor 488-labeled goat anti-rat IgG (H+L) antibody (Invitrogen) and Alexa Fluor 546-labeled goat anti-rabbit IgG (H+L) antibody (Invitrogen) were used as secondary antibodies. A fluorescence microscope (DM5500B, Leica) was used to perform visualization.

### 2. Results

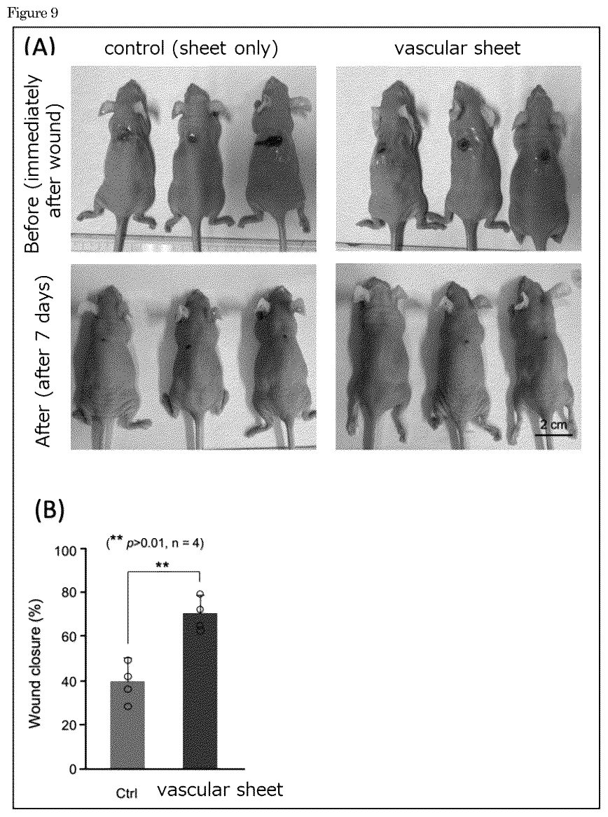

Images of the back of a nude mouse immediately after skin removal and 7 days after application of a human adipose tissue-derived vascular endothelial cell sheet are shown in FIG. 9(A). The recovery rate of the skin injury site is shown in FIG. 9(B). In the control, 40% of the injury site was covered with skin. In contrast, in the injury site where the vascular endothelial cell sheet was applied, 80% was covered with skin, indicating healing.

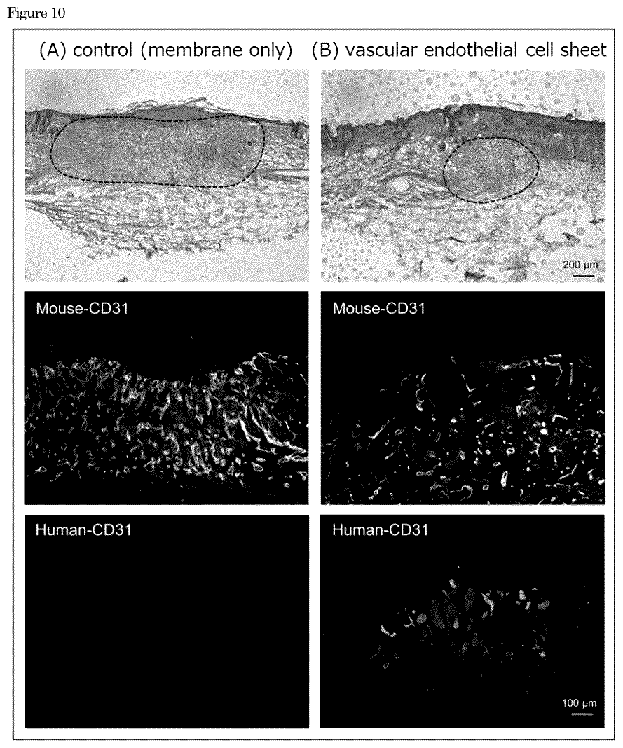

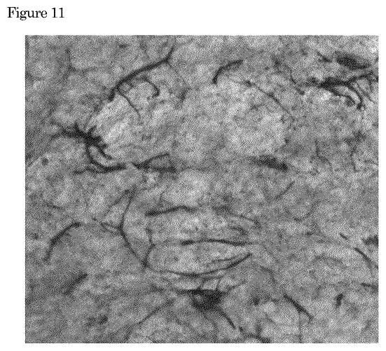

The results of histological observations are shown in FIG. 10. FIG. 10 (A) shows the results of a control mouse, and FIG. 10 (B) shows the results of a mouse with a vascular endothelial cell sheet (hereafter referred to as “cell sheet mouse”). The top row shows images stained with hematoxylin and eosin, the middle row shows images immunostained with an anti-mouse CD31 antibody, and the bottom row shows images immunostained with an anti-human CD31 antibody. In the control mouse, a large number of inflammatory cells were present at the injury site (FIG. 10 (A) upper dashed line), and epithelialization had not yet been completed. In contrast, in the cell sheet mouse, the area infiltrated by inflammatory cells was small (FIG. 10 (B) upper dashed line), and epithelialization was also completed. Furthermore, immunostaining of vascular endothelial cells (CD31-positive cells) revealed that while only mouse blood vessels were observed in the control mouse, human blood vessels were observed to have formed in the cell sheet mouse, indicating that skin damage had improved.

In this example, a sheet of vascular endothelial cells containing vascular endothelial stem cells from the underside of the porous membrane was applied to the site of skin injury. However, since it was confirmed that the cells recovered from the basement membrane matrix in Example 1 were vascular endothelial cells containing vascular endothelial stem cells, it is clear that cells recovered from the basement membrane matrix can also be used for vascular regeneration. Furthermore, cells from the underside of the porous membrane and cells from the basement membrane matrix can also be recovered and used for transplantation as dispersed cells that do not form a sheet.

### Example 3: Organ Culture of Minced Adipose Tissue Using a Collagen Membrane

### Materials and Methods

### Culture of Human Adipose Tissue

As in Example 1, human adipose tissue obtained by surgery was minced with scissors to prepare cubic minced tissue blocks with sides of approximately 2 mm. A collagen membrane (product name: Pernac, manufactured by Gunze Medical Co., Ltd.) was used as the porous membrane. Specifically, a cell strainer (manufactured by Falcon) was handmade by removing the mesh and attaching a collagen membrane to the underside. The cell strainer with the collagen membrane was inserted into a 6-well Costar cell culture multiwell plate (manufactured by Corning Incorporated) to serve as a culture vessel. Thawed basement membrane matrix (Cultrex Basement Membrane Extract (BME), R&D Systems) was supplemented with VEGF (PEPRO TECH) at a final concentration of 400 ng/ml. 2 mL of this solution was dispensed into each well of a 6-well plate and allowed to solidify at 37 °C. 3 mL of Hu-Media EG2 (serum-free liquid medium for the normal human vascular endothelial cells growth, Kurabo) was then added. Minced adipose tissue was placed on the collagen membrane of a cell strainer and inserted into each well of the 6-well plate. 1 mL of Hu-Media EG2 was added to the cell strainer, and the cells were cultured at 37 °C in 5% CO₂. The culture medium was replaced on days 6 and 12. On day 14 of culture, the cell strainer was recovered and immunostained.

### (2) Immunostaining of Cells on the Underside of the Membrane and Observation of Cells on the Matrix

To detect vascular endothelial cells on the underside of the collagen membrane, the collagen membrane was detached from the cell strainer and fixed with 4% paraformaldehyde. An anti-CD31 antibody (clone WM59, BioLegend) was used to perform immunostaining and diaminobenzidine (DAB, DOJINDO) was used to perform color development.

### 2. Results

A representative image of stained vascular endothelial cells (CD31-positive cells) on the underside of the collagen membrane is shown in FIG. 11. Even when a collagen membrane was used as the porous membrane, CD31-positive vascular endothelial cells were observed to form colonies on the underside of the membrane.

The present invention is not limited to the above-described embodiments and Examples, and various modifications are possible within the scope of the claims. Embodiments obtained by appropriately combining the technical means disclosed in the different embodiments of the present invention are also included in the technical scope of the present invention. The contents of the scientific literature and the patent literature cited herein are hereby incorporated by reference in their entirety.

1. A method for producing a vascular endothelial cell from a biological tissue or organ, comprising:
providing a first culture vessel and a second culture vessel, wherein a culture medium in the first culture vessel and a culture medium in the second culture vessel contact each other through a porous membrane having pores, which allow cell permeation, wherein a culture surface of the first culture vessel is coated with an extracellular matrix comprising a vascular endothelial cell migration factor,
culturing a biological tissue or organ in the second culture vessel, and recovering cells that have permeated through the porous membrane and migrated into the first culture vessel.

2-15. (canceled)

16. The method according to claim 1, wherein the purity of vascular endothelial cells is 50% or higher.

17. The method according to claim 16, wherein the proportion of CD157-positive cells among the vascular endothelial cells is 5% or higher.

18. The method according to claim 1, wherein the porous membrane is formed from a biocompatible material.

19. The method according to claim 18, wherein the biocompatible material is selected from the group consisting of collagen, fibrin, hyaluronic acid, polylactic acid, polyglycolic acid, polycaprolactone, polydioxanone, trimethylene carbonate, polyethylene glycol, alginate, chitosan, and mixtures thereof.

20. The method according to claim 1, wherein the vascular endothelial cell migration factor is one or more selected from the group consisting of vascular endothelial cell growth factor, basic fibroblast growth factor, hepatocyte growth factor, epidermal growth factor, SDF-1, granulocyte colony-stimulating factor, leukemia inhibitory factor, nerve growth factor, platelet-derived growth factor, stem cell growth factor, axon repulsive factor, granulocyte-macrophage colony-stimulating factor, sphingosine-1-phosphate, angiopoietin, interleukins, CCL5, CCL1, placenta growth factor, apelin, elabella, netrin, semaphorin, gremlin, BafA, and serum.

21. The method according to claim 1, wherein the extracellular matrix is one or more selected from the group consisting of basement membrane matrix, laminin, type I collagen, type IV collagen, fibronectin, vitronectin, fibrin, tenascin, hyaluronic acid, and proteoglycan.

22. A composition comprising, a vascular endothelial cell produced by the method according to claim 1.

23. A method for regenerating blood vessels, ameliorating ischemia, ameliorating malnutrition, treating vascular malformations, ameliorating blood flow insufficiency caused by vascular malformations, promoting organ regeneration, or treating diseases caused by abnormalities in molecules secreted from vascular endothelial cells comprising administering a vascular endothelial cell produced by the method according to claim 1 to a subject in need thereof.

24. A method for determining an angiogenic potential of a vascular endothelial cell produced by the method according to claim 1, comprising
placing a second culture vessel having the porous membrane on its bottom surface on top of the first culture vessel and initiating culture; and
determining that the resulting vascular endothelial cell has angiogenic potential in the case where the cells on the bottom surface of the lower culture vessel of the first culture vessel show a formation of a vascular network after culturing.

25. A method for purifying vascular endothelial cells from a biological tissue or organ, comprising:
providing a first culture vessel and a second culture vessel, wherein a culture medium in the first culture vessel and a culture medium in the second culture vessel contact each other through a porous membrane having pores, which allow cell permeation, wherein a culture surface of the first culture vessel is coated with an extracellular matrix comprising a vascular endothelial cell migration factor,
culturing a biological tissue or organ in the second culture vessel, and
recovering cells that have permeated through the porous membrane and migrated into the first culture vessel.

26. The method according to claim 25, wherein the purity of vascular endothelial cells in the recovered cells is 50% or higher.

27. A method for proliferating vascular endothelial cells from a biological tissue or organ, comprising:
providing a first culture vessel and a second culture vessel, wherein a culture medium in the first culture vessel and a culture medium in the second culture vessel contact each other through a porous membrane having pores, which allow cell permeation, wherein a culture surface of the first culture vessel is coated with an extracellular matrix comprising a vascular endothelial cell migration factor, and
culturing the biological tissue or organ in the second culture vessel.

28. A method for producing a vascular endothelial cell sheet, comprising:
providing a first culture vessel and a second culture vessel, wherein a culture medium in the first culture vessel and a culture medium in the second culture vessel contact each other through a porous membrane having pores, which allow cell permeation, wherein a culture surface of the first culture vessel is coated with an extracellular matrix comprising a vascular endothelial cell migration factor,
culturing the biological tissue or organ in the second culture vessel; and
recovering the porous membrane and removing the cells and tissue or organ present on the porous membrane on the second culture vessel side.

29. The method according to claim 28, wherein the porous membrane is formed from a biocompatible material.
